(19) United States
(12) Patent Application Publication
(10) Pub. No.: US 2009/0188490 A1
(43) Pub. Date: Jul. 30, 2009

(54) Aerosolizing Inhalation Device

(76) Inventor: Li Han, Hong Kong (CN)

Correspondence Address: HONIGMAN MILLER SCHWARTZ & COHN LLP, 38500 WOODWARD AVENUE, SUITE 100, BLOOMFIELD HILLS, MI, 48304-5048, US

(21) Appl. No.: 12/294,411

(22) PCT Filed: Nov. 9, 2007
(86) PCT No.: PCT/CN07/03180

§ 371 Date: Sep. 24, 2008

(30) Foreign Application Priority Data
Nov. 10, 2006 (CN) 200620094038.0

(51) Int. Cl. A61M 11/00

(52) U.S. Cl. 128/200.14

(57) ABSTRACT

An aerosolizing inhalation device comprising a housing including a battery, a pneumatic switch, an atomizer and a liquid-supplying bottle having a first end connected to the atomizer, wherein the liquid-supplying bottle includes a vent duct, and wherein at least a portion of the housing defines an air orifice.

### TECHNICAL FIELD

This utility model relates to an inhalation device, in particular, an aerosolizing inhalation device that aerosolizes the solution with the substance of physiologic activity into aerosol and delivers it into lungs via the respiratory tract.

### BACKGROUND

With regard to existing aerosolizing lung inhalation devices for medical purposes, a broad range of ultrasonic atomizers are available for clinic and family use. However, the diameter of aerosol particles from the existing aerosolizing devices are large, and most aerosol particles are retained by the upper respiratory tract and bronchus. As a result, they cannot effectively reach the bronchiole or alveolus. In addition, such devices are not easy to carry due to their big sizes.

### SUMMARY

In an embodiment, the invention provides an aerosolizing inhalation device, which, featuring small aerosol particles, having an aerosolizing effect, small size and portability, are suitable for outdoor medical care and rescue.

In an embodiment, the invention is directed to an aerosolizing inhalation device comprising a housing including a battery, a pneumatic switch, an atomizer and a liquid-supplying bottle having a first end connected to the atomizer, wherein the liquid-supplying bottle includes a vent duct, and wherein at least a portion of the housing defines an air orifice.

In an embodiment, the invention also adopts the following auxiliary technical solution: the air orifice is located between the said pneumatic switch and atomizer.

In an embodiment, the atomizer is a jet heating atomizer, which may include a liquid-supplying structure and heating body. In an embodiment, the liquid-supplying structure is a porous structure or fiber structure, on one end of which there is a jet hole, while on the other end, there is a protuberance. In an embodiment, the liquid-supplying bottle contacts the protuberance, so that the aerosolizing liquid can penetrate into the liquid-supplying structure. In an embodiment, the heating body is located inside the liquid-supplying structure and electrically connected with the battery.

In an embodiment, the atomizer is a impregnation heating atomizer, which includes a liquid-supplying structure and a heating body. In an embodiment, the liquid-supplying structure is a porous structure or fiber structure, one end of which forms a protuberance, while on the other end, the said heating body is installed. In an embodiment, the heating body is electrically connected with the battery. In an embodiment, the liquid-supplying bottle contacts the protuberance, so that the aerosolizing liquid can penetrate into the said liquid-supplying structure.

In an embodiment, the liquid-supplying structure is a porous structure or fiber structure. In an embodiment, the heating body may be a micro-porous ceramic framework with Ohmic metal electro-thermal material or a porous component created with electrically conductive ceramics/PTC material, which is associated with sintered electrode.

In an embodiment, the pneumatic switch is a mechanical switch made of rubber film and metal and driven by pressure difference. Alternatively, and in accordance with other embodiments, it may be a Hall element, semiconductor force-sensitive chip, silicon matrix bridge chip, capacitance or induction sensor.

In an embodiment, the liquid-supplying bottle is a porous structure or fiber structure, and is filled with aerosolizing liquid.

In an embodiment, there may be an indicator on the front end of the said housing.

In an embodiment, the invention may bring the following helpful effects:

1. In an embodiment, atomizer may be a jet heating atomizer, which carries out the primary atomization in the jet hole with the suction airflow, and then the secondary atomization by spraying onto the heating body (102). The atomization for twice enables even atomization, and small aerosolized particles with a mean diameter of about 1 μm, which is the most optimal technical parameter for lung inhalation, allowing for total inhalation.
2. In an embodiment, the atomizer has its battery, pneumatic switch, atomizer, and liquid-supplying bottle arranged in the housing in turn, featuring compact structure, small size, portability, and no restriction on application site. It is suitable for outdoor medical care and rescue.

### DESCRIPTION OF DRAWINGS

FIG. 1 is a structure diagram of an aerosolizing inhalation device, in accordance with an embodiment of the invention.

FIG. 2 is a structure diagram of a jet heating atomizer, in accordance with an embodiment of the invention.

FIG. 3 is the right view of the jet heating atomizer as shown in FIG. 2.

FIG. 4 is a structure diagram of an impregnation atomizer, in accordance with an embodiment of the invention.

FIG. 5 is the right view of the jet heating atomizer as shown in FIG. 4.

### DETAILED DESCRIPTION

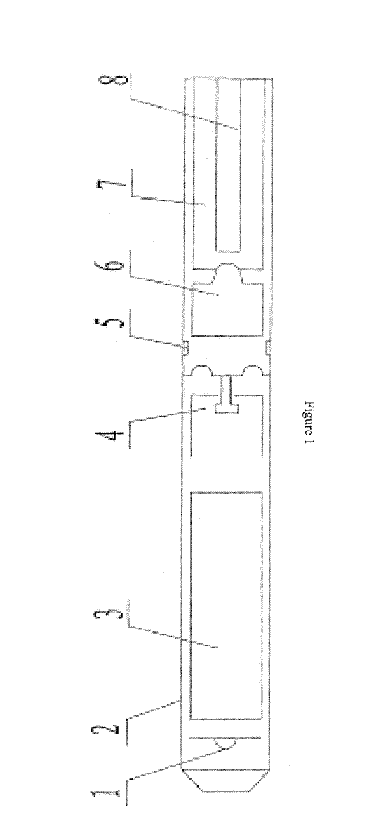

As shown in FIG. 1, in accordance with an embodiment, the invention includes a housing (2), and a battery (3), a pneumatic switch (4), an atomizer (6) and a liquid-supplying bottle (7), all of which are arranged in turn and disposed in the housing (2), which is a hollow cylinder, on the front of which is an indicator (1). The liquid-supplying bottle (7) is provided with a vent duct (8), and one end of the liquid-supplying bottle (7) is connected to the atomizer (6). On the other side of the atomizer (6), there are the pneumatic switch (4) and battery (3) successively. The battery (3), pneumatic switch (4) and atomizer (6) are in series connection to form a loop. The pneumatic switch (4) controls the operation of the atomizer (6). In this example, the pneumatic switch (4) is a mechanical switch made of rubber film and metal and driven by pressure difference. Alternatively, it may be a Hall element, semiconductor force-sensitive chip, silicon matrix bridge chip, capacitance or induction sensor. The housing (2) is provided with an air orifice (5) between the pneumatic switch (4) and atomizer (6) for air inhalation from outside.

As shown in FIGS. 2 and 3, in accordance with an embodiment of the invention, atomizer (6) is a jet heating atomizer that includes the liquid-supplying structure (101) and heating body (102). The liquid-supplying structure (101) is a porous structure or fiber structure, on one end of which there is a jet hole (103), while on the other end, there is a protuberance (104). The jet hole (103) is made of foamed ceramics, micro-porous ceramics, foamed metal, stainless steel fiber felt or chemical fiber through molding and drilling. The heating body (102) is located inside the liquid-supplying structure (101), electrically connected with the battery (3), and controlled with the pneumatic switch (4). The liquid-supplying bottle (7) contacts the protuberance (104), so that the aerosolizing liquid can penetrate into the liquid-supplying structure (101). The heating body (102) is a micro-porous ceramic framework with Ohmic metal electro-thermal material or a porous component created with electrically conductive ceramics/PTC material, which is associated with sintered electrode. The jet heating atomizer carries out the primary atomization in the jet hole (103) with the suction airflow, and then the secondary atomization by spraying onto the heating body (102). The atomization for twice enables even atomization, and small aerosolized particles with a mean diameter of about 1 μm, which is the most optimal technical parameter for lung inhalation.

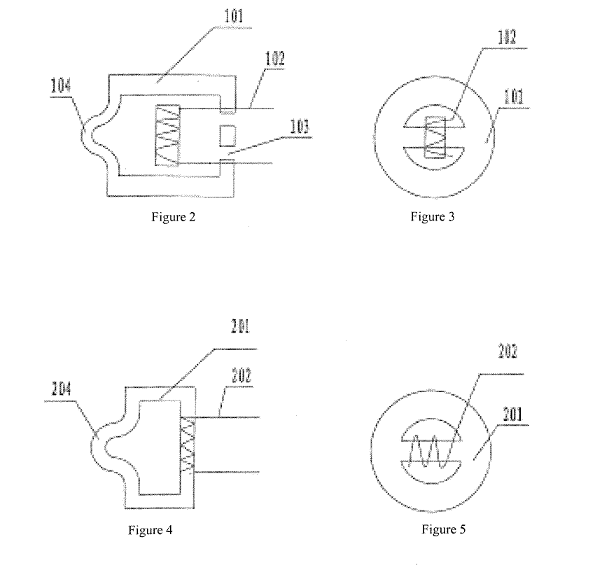

As shown in FIGS. 4 and 5, in accordance with an embodiment of the invention, atomizer (6) is an impregnation atomizer, which includes the liquid-supplying structure (201) and heating body (202). The liquid-supplying structure (201) is a porous structure or fiber structure, on one end of which is a protuberance (204), while on the other end is the heating body (202). The heating body (202) is electrically connected with the battery (3), and controlled with the pneumatic switch (4). The liquid-supplying bottle (7) contacts the protuberance (204), so that the aerosolizing liquid can penetrate into the liquid-supplying structure (201). The heating body (202) is a micro-porous ceramic framework with Ohmic metal electro-thermal material or a porous component created with electrically conductive ceramics/PTC (Positive Temperature Coefficient) material, which is associated with sintered electrode. The impregnation atomizer leverages the heating body (202) to directly heat the liquid-supplying structure (201), thus aerosolizing the solution.

In an embodiment, on one end of the atomizer (6) in the above-mentioned two examplary embodiments, the protuberances (104, 204) are connected with the liquid-supplying bottle (7). The aerosolizing solution in the liquid-supplying bottle (7) enters the atomizer (6) through the liquid-supplying structure (101, 201). The liquid-supplying structure (101, 201) is made of foamed ceramics, micro-porous ceramics, perforated metal, stainless steel fiber felt or chemical fiber through molding and drilling. The heating body (102, 202) is a micro-porous ceramic framework with Ohmic metal electro-thermal material or a porous component directly created with electrically conductive ceramics/PTC material, which is associated with sintered electrode. The surface of the heating body (101, 202) is sintered into high-temperature glaze to fix the zeolite grains, which are made of natural zeolite, artificial non-organic micro-porous ceramics or aluminum oxide grains.

In an embodiment, the invention operates as follows: The medicine is dissolved with propanediol, and the aerosolizing solution is put into the changeable liquid-supplying bottle (7). The liquid-supplying bottle (7) is put into the housing (10), contacts the atomizer (6), and supplies the liquid to it. When the user sucks, the pneumatic switch (4) accomplishes the mechanical contact under the action of pressure difference, connecting the battery (3) with the atomizer (6). The battery (3) electrifies the atomizer (6), and the heating body (102, 202) starts to heat. When the user sucks, the indicator (1) is lit, and the atomizer (6) condenses and dilutes the air in the liquid-supplying bottle (7), thus making mist, which is sucked out through the vent duct (8) on the liquid-supplying bottle.

The fiber structure for production of the liquid-supplying bottle and liquid-supplying structure is made of chemical fiber or nylon fiber.

The atomizer of this invention atomizes the aerosolizing solution impregnated from the liquid-supplying bottle, and the mist is diluted with the inhaled air into aerosol, which is inhaled by the user. In an embodiment, the invention may be used as cigarette substitute or as medical device delivering medicine to lungs.

1-11. (canceled)

12. An aerosolizing inhalation device comprising:
a housing including a battery, a pneumatic switch, an atomizer and a liquid-supplying bottle having a first end connected to said atomizer, wherein said liquid-supplying bottle includes a vent duct, and wherein at least a portion of said housing defines an air orifice.

13. The aerosolizing inhalation device of claim 12, wherein said air orifice is located between said pneumatic switch and said atomizer.

14. The aerosolizing inhalation device of claim 12, wherein said atomizer is a jet heating atomizer and includes a liquid-supplying structure and a heating body located inside the liquid supplying structure in electrical communication with said battery, wherein said liquid-supplying structure is selected from the group consisting of a porous structure and a fiber structure, and wherein the liquid supplying structure includes a first end that defines a jet hole and a second end that includes a protuberance in liquid contact with said liquid supplying bottle such that aerosolizing liquid can penetrate into said liquid-supplying structure.

15. The aerosolizing inhalation device of claim 13, wherein said atomizer is a jet heating atomizer and includes a liquid-supplying structure and a heating body located inside the liquid supplying structure in electrical communication with said battery, wherein said liquid-supplying structure is selected from the group consisting of a porous structure and a fiber structure, and wherein the liquid supplying structure includes a first end that defines a jet hole and a second end that includes a protuberance in liquid contact with said liquid supplying bottle such that aerosolizing liquid can penetrate into said liquid-supplying structure.

16. The aerosolizing inhalation device of claim 12, wherein said atomizer is an impregnation atomizer and includes a liquid-supplying structure and a heating body in electrical communication with said battery, wherein said liquid-supplying structure is selected from the group consisting of a porous structure and a fiber structure, and wherein the liquid supplying structure includes a first end that defines a jet hole and a second end that includes a protuberance in liquid contact with said liquid supplying bottle such that aerosolizing liquid can penetrate into said liquid-supplying structure.

17. The aerosolizing inhalation device of claim 13, wherein said atomizer is an impregnation atomizer and includes a liquid-supplying structure and a heating body in electrical communication with said battery, wherein said liquid-supplying structure is selected from the group consisting of a porous structure and a fiber structure, and wherein the liquid supplying structure includes a first end that defines a jet hole and a second end that includes a protuberance in liquid contact with said liquid supplying bottle such that aerosolizing liquid can penetrate into said liquid-supplying structure.

18. The aerosolizing inhalation device of claim 14, wherein said heating body is selected from the group consisting of: (i) a micro-porous ceramic framework with Ohmic metal electro-thermal material; and (ii) a porous component created with electrically conductive ceramics/PTC material, which is associated with sintered electrode.

19. The aerosolizing inhalation device of claim 15, wherein said heating body is selected from the group consisting of: (i) a micro-porous ceramic framework having Ohmic metal electro-thermal material; and (ii) a porous component created with electrically conductive ceramics/PTC material, which is associated with sintered electrode.

20. The aerosolizing inhalation device of claim 16, wherein said heating body is selected from the group consisting of: (i) a micro-porous ceramic framework having Ohmic metal electro-thermal material; and (ii) a porous component created with electrically conductive ceramics/PTC material, which is associated with sintered electrode.

21. The aerosolizing inhalation device of claim 17, wherein said heating body is selected from the group consisting of: (i) a micro-porous ceramic framework having Ohmic metal electro-thermal material; and (ii) a porous component created with electrically conductive ceramics/PTC material, which is associated with sintered electrode.

22. The aerosolizing inhalation device of claim 12, wherein the said pneumatic switch includes one or more elements selected from the group consisting of: (i) a mechanical switch made of rubber film and metal and is selectively driven by pressure difference; (ii) a Hall element; (iii) a semiconductor force-sensitive chip; (iv) a silicon matrix bridge chip; (v) a capacitance sensor; and (vi) an induction sensor.

23. The aerosolizing inhalation device of claim 13, wherein the said pneumatic switch includes one or more elements selected from the group consisting of: (i) a mechanical switch made of rubber film and metal and is selectively driven by pressure difference; (ii) a Hall element; (iii) a semiconductor force-sensitive chip; (iv) a silicon matrix bridge chip; (v) a capacitance sensor; and (vi) an induction sensor.

24. The aerosolizing inhalation device of claim 12, wherein the said liquid-supplying structure is selected from the group consisting of: (i) a porous structure and (ii) a fiber structure, wherein said porous structure or said fiber structure includes aerosolizing solution.

25. The aerosolizing inhalation device of claim 13, wherein the said liquid-supplying structure is selected from the group consisting of: (i) a porous structure and (ii) a fiber structure, wherein said porous structure or said fiber structure includes aerosolizing solution.

26. The aerosolizing inhalation device of claim 18, wherein the said liquid-supplying structure is selected from the group consisting of: (i) a porous structure and (ii) a fiber structure, wherein said porous structure or said fiber structure includes aerosolizing solution.

27. The aerosolizing inhalation device of claim 19, wherein the said liquid-supplying structure is selected from the group consisting of: (i) a porous structure and (ii) a fiber structure, wherein said porous structure or said fiber structure includes aerosolizing solution.

28. The aerosolizing inhalation device of claim 20, wherein the said liquid-supplying structure is selected from the group consisting of: (i) a porous structure and (ii) a fiber structure, wherein said porous structure or said fiber structure includes aerosolizing solution.

29. The aerosolizing inhalation device of claim 21, wherein the said liquid-supplying structure is selected from the group consisting of: (i) a porous structure and (ii) a fiber structure, wherein said porous structure or said fiber structure includes aerosolizing solution.

30. The aerosolizing inhalation device of claim 28, wherein the said liquid-supplying structure is selected from the group consisting of: (i) a porous structure and (ii) a fiber structure, wherein said porous structure or said fiber structure includes aerosolizing solution.

31. The aerosolizing inhalation device of claim 29, wherein the said liquid-supplying structure is selected from the group consisting of: (i) a porous structure and (ii) a fiber structure, wherein said porous structure or said fiber structure includes aerosolizing solution.
